(19) United States
(12) Patent Application Publication
(10) Pub. No.: US 2023/0258346 A1
(43) Pub. Date: Aug. 17, 2023

(54) GAS PURIFICATION DEVICE, AIR-CONDITIONING TERMINAL PRODUCT AND AIR-CONDITIONING UNIT

(71) Applicant: Carrier Corporation, Palm Beach Gardens, FL (US)

(72) Inventors: Huanan Li, Shanghai (CN); Ran Yang, Shanghai (CN); Shuguang Zhang, Shanghai (CN); Huaxi Li, Shanghai (CN)

(21) Appl. No.: 18/166,009

(22) Filed: Feb. 8, 2023

(30) Foreign Application Priority Data
Feb. 11, 2022 (CN) 202210128729.1

(51) Int. Cl. F24F 8/30; F24F 1/0076; A61L 9/22

(52) U.S. Cl. CPC F24F 8/30; F24F 1/0076; A61L 9/22; A61L 2209/16

(57) ABSTRACT

A gas purifying device for an air-conditioning terminal product includes a housing; an ion generator secured to the housing; blades arranged on the outer side of the housing and used for receiving wind power from the air-conditioning terminal product for rotation; and an electric generator arranged in the housing and connected to the blades through a rotor shaft; when rotating, the blades drive the electric generator to operate to convert wind energy into electric energy, thereby enabling the electric generator to supply power to the ion generator. An air-conditioning terminal product provided with the gas purification device, and an air-conditioning unit configured with the air-conditioning terminal product.

### FOREIGN PRIORITY

This application claims priority to Chinese Patent Application No. 202210128729.1, filed Feb. 11, 2022, and all the benefits accruing therefrom under 35 U.S.C. § 119, the contents of which in its entirety are herein incorporated by reference.

### FIELD OF THE INVENTION

The present invention relates to the technical field of gas purification, in particular to a gas purification device for an air-conditioning terminal product, and also relates to an air-conditioning terminal product provided with the gas purification device and an air-conditioning unit configured with the air-conditioning terminal product.

### BACKGROUND OF THE INVENTION

In the current COVID-19 pandemic, the generally accepted mechanism of epidemic transmission is that viruses are mainly transmitted through the droplets of the infected people while they are coughing, sneezing and talking. Most viruses are nano-particles that easily form aerosols and remain drifting in the air, which are infectious until inactive. Because of these characteristics, people nowadays pay more and more attention to air purification and sterilization, while ordinary air purifiers with air filtration function only can no longer meet the needs of the public. Therefore, products for air purification using positive and negative ion generators have gradually entered the market and have a good development prospect. The positive and negative ion generator is a generator that produces positive and negative ions. In this generator, low voltage is raised to positive high voltage and negative high voltage through a booster circuit, and air is ionized under the action of the positive high voltage electric field and the negative high voltage electric field, thereby generating a large number of positive and negative ions. The positive and negative oxygen ions emitted into the air attract each other in pairs. During the process of mutual attraction, the positive and negative oxygen ions meet with the airborne bacteria in the air. Enormous energy is released at the moment when the positive and negative charges are neutralized, which results in the change of structure and transfer of energy of the surrounding bacteria, thus killing the airborne bacteria.

### SUMMARY OF THE INVENTION

In view of the foregoing, according to a first aspect of the present invention, a gas purification device for an air-conditioning terminal product is provided. In the gas purification device according to the present invention, the gas purification device comprises: a housing; an ion generator, secured to the housing; blades, arranged on the outer side of the housing, and used for receiving wind power from the air-conditioning terminal product for rotation; and an electric generator, arranged in the housing and connected to the blades through a rotor shaft, wherein, when rotating, the blades drive the electric generator to operate to convert wind energy into electric energy, thereby enabling the electric generator to supply power to the ion generator.

In another embodiment of the gas purification device according to the present invention, the gas purification device further comprises an adjustable fixed bracket for securing the housing of the gas purification device in the ventilation circuit of the air-conditioning terminal product.

In yet another embodiment of the gas purification device according to the present invention, the fixed bracket comprises: a first support element fixedly connected to the housing of the gas purification device; and a second support element movable along the length direction of the first support element, wherein, the first support element and the second support element are bolted to the air-conditioning terminal product.

In still another embodiment of the gas purification device according to the present invention, three of the fixed brackets are provided, and the three fixed brackets are arranged on the housing at equal intervals of 120 degrees, centered on the axis of the rotor shaft.

In another embodiment of the gas purification device according to the present invention, the first support element of the fixed bracket and the housing are made of plastic and are integrally formed.

In yet another embodiment of the gas purification device according to the present invention, the housing of the gas purification device is provided with an indicator lamp for displaying the operating state.

In still another embodiment of the gas purification device according to the present invention, the ion generator comprises paired positive and negative ion generators.

In a further embodiment of the gas purification device according to the present invention, the emitter of the ion generator is a carbon brush or a steel needle.

In still a further embodiment of the gas purification device according to the present invention, the gas purification device also comprises a cleaning element for wiping the emitter of the ion generator, wherein the cleaning element is powered by the electric generator.

In addition, according to a second aspect of the present invention, an air-conditioning terminal product is also provided, wherein the air-conditioning terminal product is provided with the aforementioned gas purification device and comprises a fan coil.

In yet another embodiment of the air-conditioning terminal product according to the present invention, the fan coil comprises a first centrifugal fan and a second centrifugal fan arranged coaxially and driven by the same motor, wherein the ventilation circuits of the first centrifugal fan and the second centrifugal fan are respectively provided with the gas purification device.

Furthermore, according to a third aspect of the present invention, an air-conditioning unit is further provided, wherein the air-conditioning unit is configured with the aforementioned air-conditioning terminal product.

It would be appreciated that the gas purification device for an air-conditioning terminal product according to the present invention adopts the design of wind power generation, which can not only achieve the goal of zero energy consumption, but also effectively purify the dust, harmful gas, odor and bacteria in the environment.

### BRIEF DESCRIPTION OF THE DRAWINGS

The technical solutions of the present invention will be described in further detail below in conjunction with the accompanying drawings and embodiments, where:

FIG. 1 is a structural schematic diagram of an embodiment of a gas purification device for an air-conditioning terminal product according to the present invention;

FIG. 2 is an exploded diagram of the gas purification device for an air-conditioning terminal product in FIG. 1; and

FIG. 3 is a structural schematic diagram of the gas purification device for an air-conditioning terminal product in FIG. 1 when installed on an air-conditioning terminal product.

### DETAILED DESCRIPTION OF THE EMBODIMENT(S) OF THE INVENTION

Several embodiments of the present invention will be described in detail below in conjunction with the accompanying drawings. The described embodiments are only examples of the present invention. In addition, for the sake of brevity of these embodiments, all features actually realized may not be fully described in the description.

In the depiction of the embodiments of the present invention, it is to be appreciated that the directional or positional relationships indicated by the terms “center”, “longitudinal”, “transverse”, “up”, “down”, “front”, “rear”, “left”, “right”, “vertical”, “horizontal”, “top”, “bottom”, “inside”, “outside” and the like are based on the directional or positional relationships shown in the accompanying drawings, which are intended only to facilitate and simply the description of the present invention, but not to indicate or imply that the devices or components must have a specific direction or be constructed and operated in a specific direction. Therefore, they cannot be understood as limiting the present invention.

In addition, the terms “first”, “second”, “third” and the like are used only for descriptive purposes and cannot be understood as indicating or implying the relative importance thereof or impliedly indicating the number of technical features indicated. Thus, features that are limited by the terms “first”, “second” or the like may include, expressly or implicitly, one or more of such features. In the depiction of the present invention, unless otherwise stated, “a plurality of” means two or more.

The terms “install”, “connected”, “connect” should be interpreted in a broad sense. For example, it can be a fixed connection, a detachable connection, or an integral connection; or it can be a mechanical connection or an electrical connection; or it can be directly connected, indirectly connected through an intermediate medium, or connected within two components. To those skilled in the art, the specific meaning of the above terms in the present invention may be interpreted on a case-by-case basis.

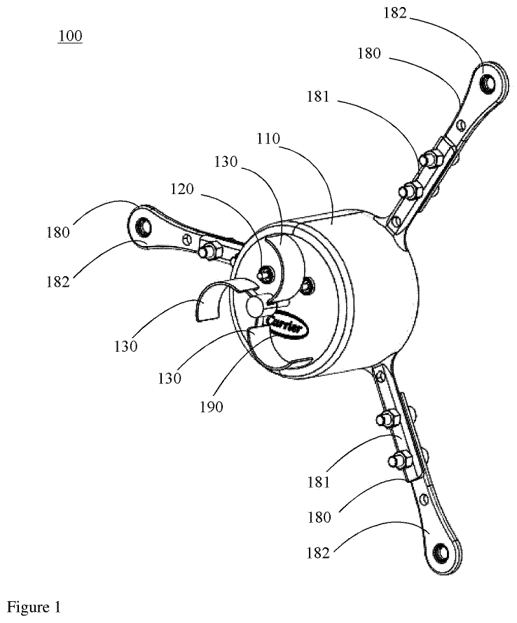

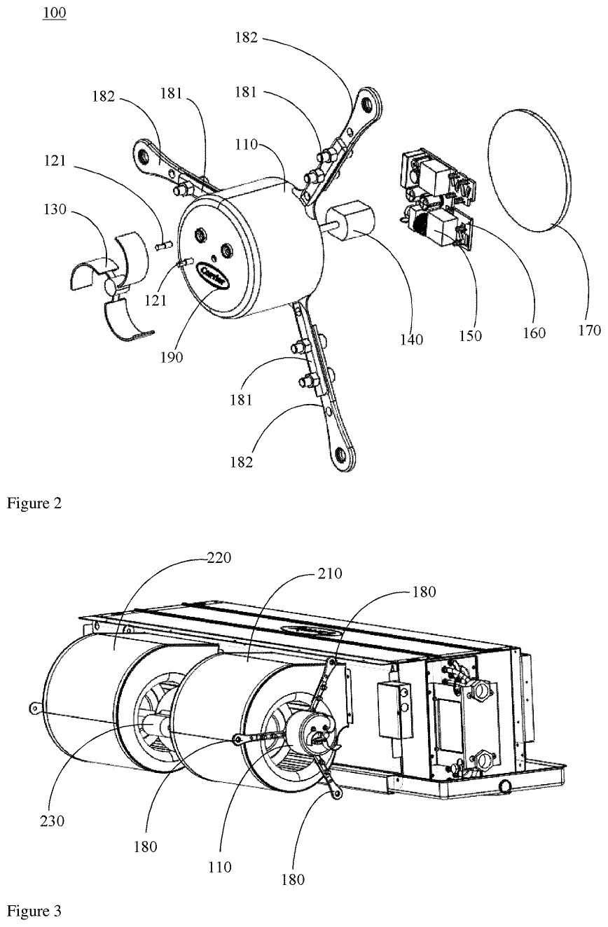

As shown in FIG. 1, it schematically illustrates in general the structure of an embodiment of the gas purification device for an air-conditioning terminal product according to the present invention. As can be explicitly seen from FIGS. 1 and 2, the gas purification device 100 mainly consists of a housing 110, an ion generator 120, blades 130, an electric generator 140, an electric component 150, a printed circuit board 160, a rear end cover 170, and other components. The ion generator 120 is secured to the housing 110, where the emitter 121 of the ion generator 120 protrudes outward from the housing 110. The emitter 121 is, for example, a carbon brush or a steel needle, where the carbon brush is a release tip made of element carbon and is used to generate positive or negative ions, so as to play the role of removing dust, sterilizing and purifying air etc. The blades 130 are arranged on the outer side of the housing 110 to receive wind force from the air-conditioning terminal product for rotation. The blades 130 are in the form of a wind blade set similar to an electric fan and are made of plastic, metal or other materials, the specific installation form of which depends on the size of the air-conditioning terminal product and the condition of the installation space. For example, the blades 130 can be installed above the emitter 121 of the ion generator 120, so as to prevent the blades 130 from scratching the emitter 121 of the ion generator 120 due to excessively high rotational frequency. In addition, the electric generator 140, the electric component 150, and the printed circuit board 160 are all arranged in the housing 110, and the electric generator 140 is connected to the blades 130 through the rotor shaft. When rotating, the blades 130 drive the electric generator 140 to operate to convert wind energy into electric energy, thereby enabling the electric generator 140 to provide electric energy for the ion generator 120. Due to the design of wind power generation, the gas purification device of the present invention does not need additional external power supply that are expensive, thus greatly saving the cost of installing and maintaining the external power supply and advantageously realizing the goal of zero energy consumption.

In conjunction with the above embodiment, in other preferred embodiments, in order to advantageously secure the gas purification device at a target position, such as in the ventilation circuit of the air-conditioning terminal product, the gas purification device 100 also comprises adjustable fixed brackets 180. Specifically, the fixed bracket 180 may comprise a first support element 181 and a second support element 182, which are provided with several through holes respectively. The support element 181 is fixedly connected to the housing 110 of the gas purification device 100. For ease of manufacture, the first support element 181 of the fixed bracket 180 and the housing 110 can be made of plastic and integrally formed. The second support element 182 is movable along the length direction of the first support element 181, and the first support element 181 and the second support element 182 are bolted to the air-conditioning terminal product, for example to the volute of a centrifugal fan. Further, three of the fixed brackets 180 are provided, which are arranged on the housing 110 at equal intervals of 120 degrees, centered on the axis of the rotor shaft, as shown in FIG. 1. It should be noted that the number of fixed brackets is not limited to three, but can be one, two, four, five or more, as long as the gas purification device can be securely fixed in the ventilation circuit of the air-conditioning terminal product. The structure of the adjustable fixed bracket is not limited to the above form, as long as the fixed bracket can satisfy different types of air-conditioning terminal products. Furthermore, in addition to the above adjustable fixed brackets, the gas purification device can be secured in the ventilation circuit of the air-conditioning terminal product in other detachable ways.

With continued reference to FIGS. 1-2, the ion generator of the gas purification device comprises paired positive and negative ion generators. It can be appreciated by those skilled in the art that the paired positive and negative ion generators can form a bipolar ion generator, so as to achieve a good sterilization and disinfection effect. The bipolar ion generator generally uses O₂⁺ and O₂⁻ produced by corona discharge to purify air. O₂⁺ and O₂⁻ produced by corona discharge or other methods have very high chemical activity and can react with water molecules in the air to form H₂O₂. In the process of further chemical reaction, the oxidant decomposes bacterial protein, thus achieving the disinfection and sterilization effect. Therefore, in a closed environment, the gas purification device of the present invention can inactivate microorganisms and bacteria and effectively inhibit their growth.

It is understandable, of course, that the number, location and specification of the carbon brushes of the positive ion generators and the negative ion generators can be different, so as to meet the purification needs of different environments. As an alternative, the ion generator can be designed to contain only negative ion generators, or more negative ion generators than positive ion generators. Such a design may enable the gas purification device to produce negative ions only, or enable the gas purification device to produce more negative ions than positive ions. It is noteworthy that the content of negative ions in the air is an important factor to determine the air quality. When more negative ions are contained in the air, it is conducive to more effective dust removal, sterilization and air purification, and is also beneficial to human health.

As shown in FIGS. 1 and 2, the housing 110 of the gas purification device 100 is further provided with an indicator lamp 190 for displaying the operating state. The indicator lamp 190 can also be powered by the electric generator 140. When the gas purification device 100 is in operation, i.e., when the blades 130 drive the electric generator 140 to operate under the action of wind force, the indicator lamp 190 powered by the electric generator 140 lights up, thus indicating that the electric generator 140 is functioning properly.

In some embodiments, the gas purification device 100 may also comprise a cleaning element (not shown) for wiping the emitter 121 of the ion generator 120. The cleaning element can be in the form of a rotating blade assembly or a linearly reciprocating cleaning arm, and is powered by the electric generator 140. The cleaning element wipes the emitter 121 by rotational movement or linear reciprocating movement, thereby preventing dust deposition on the emitter 121 and thus extending the service life of the ion generator 120.

In addition, the present invention also provides an air-conditioning terminal product. The air-conditioning terminal product is provided with the aforementioned gas purification device 100, and comprises a fan coil 200. As shown in FIG. 3, the fan coil 200 comprises a first centrifugal fan 210 and a second centrifugal fan 220, where the first centrifugal fan 210 and the second centrifugal fan 220 are arranged coaxially and can be driven by the same motor 230, thus forming a “one to two” structure. The gas purification device according to the present invention is flexible in arrangement and easy to disassemble. For example, the aforementioned gas purification device can be respectively arranged in the ventilation circuits of the first centrifugal fan 210 and the second centrifugal fan 220 of the fan coil 200, thus achieving the purpose of sterilization and air purification. Of course, the air-conditioning terminal product can comprise four centrifugal fans, where every two centrifugal fans are arranged coaxially and driven by the same motor, thus forming a “two to four” structure. Each ventilation circuit of the four centrifugal fans is arranged with the aforementioned gas purification device respectively.

Furthermore, the present invention further provides an air-conditioning unit. The air-conditioning unit is configured with the aforementioned air-conditioning terminal product. As is well known to those skilled in the art, in addition to the aforementioned air-conditioning terminal product, the air-conditioning unit generally also comprises cooling/heating units, and the like.

In summary, the air purification device for an air-conditioning terminal product according to the present invention adopts the design of wind power generation, which can not only advantageously achieve the goal of zero energy consumption, but also effectively purify the dust, harmful gas, odor and bacteria in the environment. Therefore, the gas purification device is very suitable for large-scale production, manufacturing and application as a general-purpose device installed in air-conditioning terminal products.

Some specific embodiments are listed above to illustrate in detail the gas purification device for an air-conditioning terminal product, the air-conditioning terminal product provided with the gas purification device, and the air-conditioning unit configured with the air-conditioning terminal product according to the present invention. These individual examples are only used to illustrate the principle of the present invention and the implementations thereof, but not to limit the present invention. Those skilled in the art may, without departing from the spirit and scope of the present invention, make various modifications and improvements. Therefore, all equivalent technical solutions shall belong to the scope of the present invention and be defined by the various claims of the present invention.

What is claimed is:

1. A gas purification device for an air-conditioning terminal product, comprising:
a housing;
an ion generator, secured to the housing;
blades, arranged on an outer side of the housing, and used for receiving wind power from the air-conditioning terminal product for rotation; and
an electric generator, arranged in the housing and connected to the blades through a rotor shaft,
wherein, when rotating, the blades drive the electric generator to operate to convert wind energy into electric energy, thereby enabling the electric generator to supply power to the ion generator.

2. The gas purification device according to claim 1, wherein the gas purification device further comprises an adjustable fixed bracket for securing the housing of the gas purification device in a ventilation circuit of the air-conditioning terminal product.

3. The gas purification device according to claim 2, wherein the fixed bracket comprises:
a first support element, fixedly connected to the housing of the gas purification device; and
a second support element, movable along a length direction of the first support element,
wherein, the first support element and the second support element are bolted to the air-conditioning terminal product.

4. The gas purification device according to claim 2, wherein three of the fixed brackets are provided, and the three fixed brackets are arranged on the housing at equal intervals of 120 degrees, centered on an axis of the rotor shaft.

5. The gas purification device according to claim 3, wherein the first support element of the fixed bracket and the housing are made of plastic and are integrally formed.

6. The gas purification device according to claim 1, wherein the housing of the gas purification device is provided with an indicator lamp for displaying an operating state.

7. The gas purification device according to claim 1, wherein the ion generator comprises paired positive and negative ion generators.

8. The gas purification device according to claim 1, wherein an emitter of the ion generator is a carbon brush or a steel needle.

9. The gas purification device according to claim 8, wherein the gas purification device further comprises a cleaning element for wiping the emitter of the ion generator, and the cleaning element is powered by the electric generator.

10. An air-conditioning terminal product, wherein the air-conditioning terminal product is provided with the gas purification device according to claim 1, and comprises a fan coil.

11. The air-conditioning terminal product according to claim 10, wherein the fan coil comprises a first centrifugal fan and a second centrifugal fan arranged coaxially and driven by the same motor, and ventilation circuits of the first centrifugal fan and the second centrifugal fan are respectively provided with the gas purification device.

12. An air-conditioning unit, wherein the air-conditioning unit is configured with the air-conditioning terminal product according to any of claim 10.
